(19) United States
(12) Patent Application Publication
(10) Pub. No.: US 2007/0112426 A1
(43) Pub. Date: May 17, 2007

(54) Thin flexible intraocular implant

(76) Inventors: Francois Franceschi, Marseille (FR); Cyrille Vinchon, Chilly (FR)

Correspondence Address: COHEN, PONTANI, LIEBERMAN & PAVANE, 551 FIFTH AVENUE, SUITE 1210, NEW YORK, NY, 10176, US

(21) Appl. No.: 10/566,228

(22) PCT Filed: Jul. 22, 2004
(86) PCT No.: PCT/FR04/50351

§ 371 Date: Jan. 27, 2006

(30) Foreign Application Priority Data
Aug. 4, 2003 (FR) 03 09595

(51) Int. Cl. A61F 2/16

(52) U.S. Cl. 623/6.47; 623/6.44

(57) ABSTRACT

A capsular bag intraocular implant having an optic portion and a haptic portion with the haptic portion having at least two arms extending radially relative to the optic portion. Each arm of the haptic portion has a main portion, a connection end connected to the edge of the optic portion and having a thickness in the direction of the optical axis that is less than the thickness of the main portion so as to form a flexing line that is substantially tangential to the optic portion, and a contact end presenting a contact edge against the inside wall of the capsular bag. The contact edge is disposed on a circle that is concentric with the optic portion and of diameter not less than 10.5 mm. The main portion of each arm forms an angle in a forward direction relative to the optical plane.

The present invention relates to a flexible intraocular implant of small thickness.

More precisely, the invention relates to an intraocular implant in which the thickness of the optic portion is reduced while nevertheless presenting mechanical properties that are sufficient for ensuring that the optic portion is properly held in the capsular bag.

It is known that intraocular implants are usually used for replacing the natural lens after it has been removed during a cataract operation. The surgical techniques that are presently available, in particular for removing the natural lens by phaco-emulsification, make it possible to perform the operation while making an incision in the cornea that is very small, about 3 millimeters (mm). It is therefore advantageous to have intraocular implants that can be inserted into the capsular bag through an incision presenting this same small size of about 3 mm. It is known that the smaller the incision, the faster the eye heals.

As is well known, an intraocular implant comprises firstly an optic portion constituting the optical system for correcting the revision, and secondly by a haptic portion which serves to hold the optic portion in the capsular bag.

In order to enable the implant to be inserted into the eye through an incision of small size, it is necessary to be able to fold at least the optic portion of the implant, since its diameter, which needs to be at least about 5 mm, is clearly much greater than the size of the incision. In order to allow this optic portion to be folded, it is necessary to use a “flexible” material such as a hydrophilic acrylic or a hydrogel or some other similar substance. It is also necessary for the optic portion to be of thickness that is as small as possible. The thickness of the optic portion on its optical axis is the result firstly of the radii of curvature of the anterior and posterior interface surfaces and secondly the thickness of the edge of said optic portion, which thickness must be sufficient to enable the haptic portion to be secured to the periphery of the optic portion.

In order to increase the radii of curvature of the interface surfaces, and thus reduce the thickness of the optic, it is possible to use transparent materials presenting a refractive index that is high. Nevertheless it is not possible to use materials presenting very high indices since it is well known that they induce reflection phenomena that disturb the vision of the patient fitted with the implant.

In order to reduce thickness, it is therefore necessary to reduce as much as possible the edge thickness of the optic portion, which naturally leads to a decrease in the thickness of the-haptic portion, at least in its connection zone. It will readily be understood that reducing the thickness of the haptic portion, while also using a flexible material, naturally leads to the haptic portion having mechanical properties that are mediocre.

As explained above, the function of the haptic portion is to hold the optic portion on the optical axis of the eye and to avoid any axial movements of said optic portion.

An object of the present invention is to provide an intraocular implant made using a flexible material that presents improved antero-posterior axial stability while also enabling an implant to be made with an optic portion that presents small thickness.

The capsular bag intraocular implant comprises:

an optic portion of substantially circular shape presenting an edge, and anterior and posterior interface surfaces; and

a haptic portion comprising at least two arms extending radially relative to the optic portion, said implant being characterized in that each arm comprises:
a main portion;
a connection end connected to the optic portion, said connection end having a thickness in the direction of the optical axis that is smaller than the thickness of the main portion so as to form a flexing line that is substantially tangential to the optic portion; and
a contact end presenting a contact edge for contacting the inside wall of the capsular bag; said contact edge being disposed on a circle that is concentric about the optic portion and of diameter greater than the diameter of the capsular bag, being not less than 10.5 mm;
said main portion of each arm forming an angle in a forward direction relative to the optical plane in such a manner that said flexing line is closer to the optical plane than is said contact edge;

whereby, when the implant is put into place in the capsular bag, the optic portion is displaced towards the posterior wall of the capsular bag by the arms turning about the flexing lines defined by their connection ends, under the effect of the stress applied by the capsular bag to the contact ends of the arms.

It will be understood that by means of the invention, because the connection zone between the haptic arms and the optic portion is very small in section, and because the outside diameter of the haptic portion is very substantially greater than the inside diameter of the capsular bag, the deformation of the haptic portion takes place in the form of the arms turning about the connection zones because of their small section. This turning moves the optic portion in stable manner towards the posterior wall of the capsular bag, in particular because the particular angle given to the haptic arms before they are deformed causes the contact edges to be “in front” of the flexing lines. This ensures that the optic portion is well stabilized by the posterior interface surface pressing against the posterior wall of the capsular bag. In addition, the posterior projection that leads to intimate contact between the posterior interface surface and the posterior capsule associated with effective force, avoids or substantially limits any risk of cells proliferating on the posterior portion of the capsular bag, which would naturally disturb the vision of the person fitted with the implant.

In addition, because of the reduced thickness of the haptic arms, it should be added that the square edge exists over the entire periphery of the optic portion, including in the connection zones of the haptic arms at the periphery of the optic portion.

In a preferred embodiment, the intraocular implant is characterized in that the contact end of each arm is bent rearwards relative to the main portion of the arm forming a bend in such a manner that the contact terminal or end portion is closer to the optical plane of the optic portion than is the bend, whereby, under the effect of the stress applied by the capsular bag, the connection ends come to bear against the anterior wall of the capsular bag.

In this preferred embodiment, the rearward projection of the optic portion of the implant is made even more stable because the contact ends of the arms are angled relative to the main portions thereof. These end portions come to bear against the anterior face of the capsular bag, and the main portions of the arms behave like pillars extending substantially orthogonally to the optic portion and they hold it in stable manner against the posterior wall of the capsular bag.

Also preferably, the intraocular implant is characterized in that it further comprises at least two connection pieces in the form of circular arcs concentric with the optic portion, the end of each connection piece being connected to an arm, said connection pieces lying on the same circle.

The circularly arcuate connection pieces present between the haptic arms and concentric about the optic portion provide mutual stabilization of the arms while avoiding any risk of said arms flexing in their connection zones about pivot axes parallel to the optical axis.

Other characteristics and advantages of the invention appear better on reading the following description of a plurality of embodiments of the invention given as non-limiting examples.

The description refers to the accompanying figures, in which:

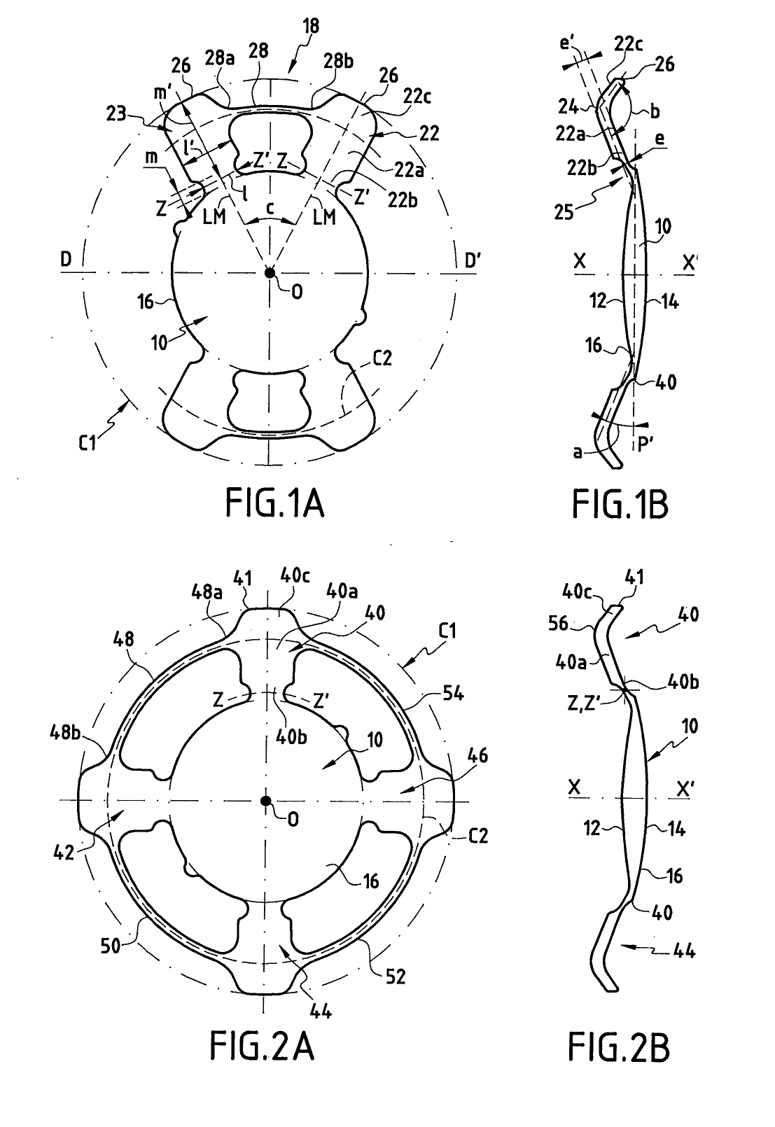

FIG. 1A is a face view of a first embodiment of the implant;

FIG. 1B is a side view of the FIG. 1A implant;

FIG. 2A is a face view of a second embodiment of the implant of the invention;

FIG. 2B is a side view of the FIG. 2A implant;

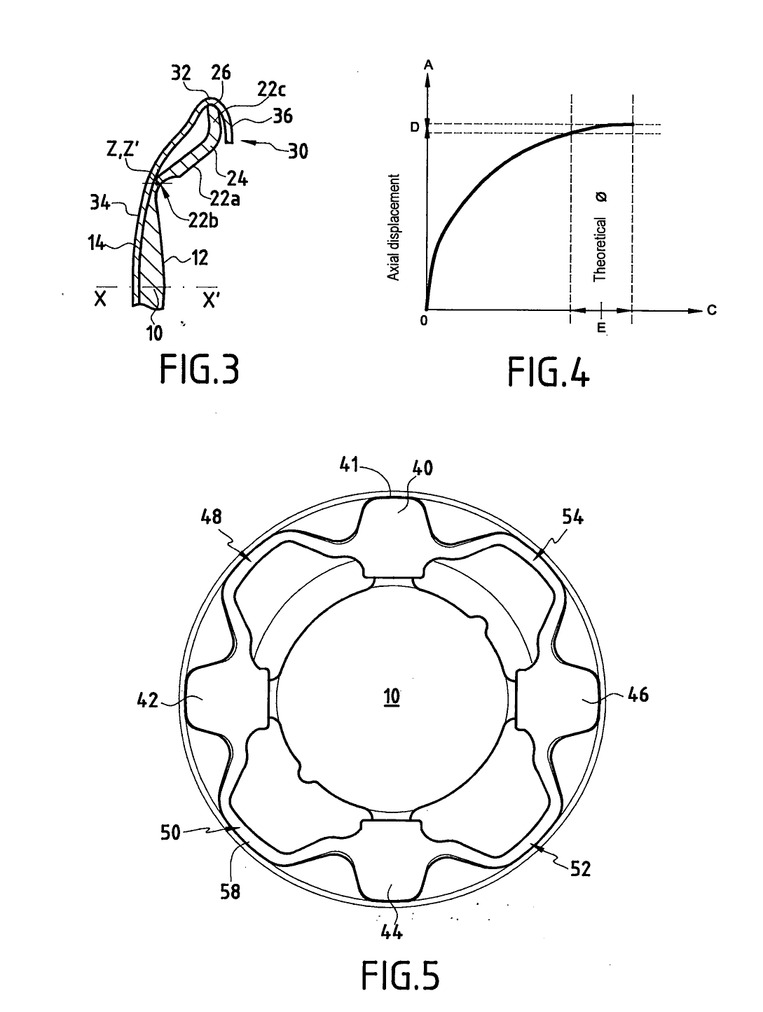

FIG. 3 is a fragmentary vertical section view showing the deformation of the haptic arms of the implant when the implant is put into place in the capsular bag;

FIG. 4 is a graph showing the axial displacement of the implant as a function of the compression of the haptic portion;

FIG. 5 is a face view of the FIG. 2A implant when put into place in the capsular bag;

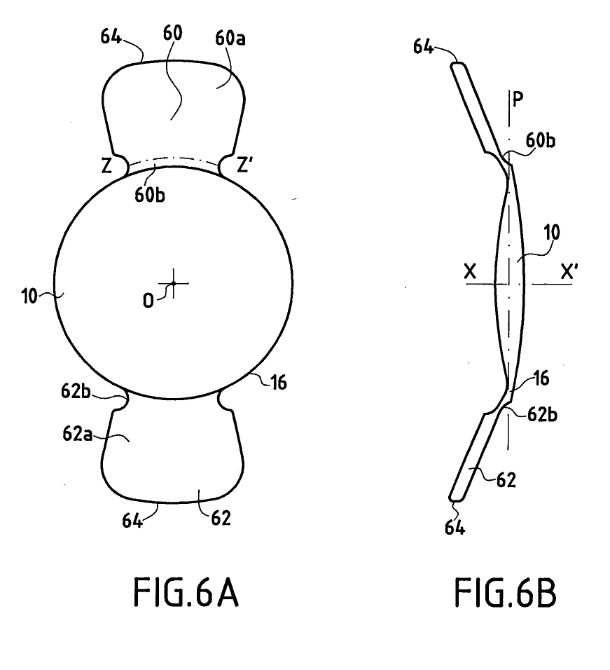

FIG. 6A is a face view of a third embodiment of the implant; and

FIG. 6B is a side view of the FIG. 6A implant.

A first embodiment of the intraocular implant of the invention is described initially, and with reference to FIGS. 1A and 1B.

The implant is constituted by an optic portion 10 presenting an anterior interface surface 12, a posterior interface surface 14, and a peripheral edge 16. This optic is substantially circular.

In this embodiment, the haptic portion is constituted by two sets of haptics given respective references 18 and 20. These two sets of haptics 18 and 20 are identical and symmetrical about the diameter D-D′ of the optic portion 10. Only the haptic set 18 is therefore described in detail.

The haptic set 18 is constituted by two radial arms 22 and 23 having mean lines LM projected onto the optical plane P-P′ that extend radii of the optic portion 10. These mean lines LM form between them an angle c of less than 90°, e.g. of 60°. Each haptic arm 22 or 23 presents a main portion 22a, a connection end 22b at the periphery 16 of the optic portion, and a contact end 22c for coming into contact with the wall of the capsular bag when the implant is put into place therein. The mean line of the contact end 22c is substantially rectangular. The connection end 22b presents a right section that is much smaller than that of the main portion 22a of the arm, being equal to no more than half of it, the main portion 22a of the arm having a right section that is substantially constant. In particular, the width l of the connection end is less than the width l′ of the main portion of the arm, and its thickness e is much less than the thickness e′ of the main portion of said arm. This reduction in thickness is such as to define a posterior “step” 25 that forms a “square edge” with the posterior interface surface in the connection zone between the optic portion and the arms. In addition, the length m of the connection end 22b, along its mean line, is much shorter than the length m′ of the set of arms. The connection end 22b thus defines a flexing or pivoting line Z-Z′ for the arm relative to the optic portion, which line is substantially tangential to the periphery 16 of the optic portion under the effect of the continuities applied to the contact ends. In addition, it will be understood that the arm has a single flexing line which is defined by the connection end 22b.

FIG. 1B shows that the mean line LM of the main portion 22a of the arm 22, which line is substantially rectilinear, forms an angle a relative to the optical plane P-P′, and lies in front of said plane. This angle a is preferably not less than 5°. In other words, the main portion of the arm lies in the same half-space defined by the plane P-P′ as does the anterior interface surface 12 of the optic portion. FIG. 1B also shows that the contact end 22c of the arm 22 forms an angle b relative to the main portion 22a of said arm. The contact end 22c is thus “directed rearwards”. If the bend between the contact end 22c and the main portion 22a is referenced 24, then the angle b, which lies in the range 90° to 150° and is preferably equal to 120°, is such that the contact end, and more precisely its contact edge 26, is closer to the optical plane P-P′ than is the bend 29. In any event, the angles a and b should be determined so that the contact edges 26 of the arm lie “in front” of the flexing lines Z-Z′, i.e. that the flexing lines should be closer to the optical plane P-P′ than are the contact edges 26, and so that the contact edges lie in the same half-space defined by the optical plane P-P′ as contains the anterior interface surface. The mean lines of the main portion 22a and of the contact end 22c of any one arm lie substantially in the same plane containing the optical axis of the implant.

In other words, the angle between the optical plane P-P′ and the line joining the contact edge 26 to the flexing line is not less than 1° forwards.

Furthermore, in the region of the bend 24, the arm 22 is of a thickness that is much greater than its thickness at its connection end 22b. Under the effect of the stresses applied to the arm, deformation will be localized in the connection zone 22b and will leave the bend 24 unaffected, i.e. it will remain undeformed.

The contact edge 26 of each haptic arm is substantially in the shape of an arc of a circle having a diameter lying in the range 2.5 mm to 10.5 mm, and preferably equal to 10 mm, which corresponds to the diameter of the capsular bag.

In addition to the radial arms 22 and 23, the haptic set 18 preferably also includes a link piece 28 in the form of an arc of a circle that is concentric with the optic portion 10. The link piece 28 presents two ends 28a and 28b where it is secured to the arms 22 and 23. The secured ends 22a and 22b are preferably situated level with the bend 24 in each of the arms 22 and 23.

The terminal contact edges 26 of the contact ends 22c and 23c of the arms lie on a circle C1 of center O having a diameter that is substantially greater than the diameter of the capsular bag. The diameter of the circle C1 is not less than 10.5 mm and preferably lies in the range 11 mm to 11.5 mm. In addition, the bends 24 of the arms and the link piece 28 lie on a circle C2 of center O and of diameter equal to 10 mm, for example, i.e. substantially the diameter of the capsular bag.

With reference now to FIG. 3, there follows an explanation of how the implant behaves while being put into place in the capsular bag. This figure shows the capsular bag 30 with its equatorial zone 32, its posterior capsule 34, and the residual portion 36 of its anterior capsule. When the implant is put into place in the capsular bag 30, the bag, and more precisely its equatorial zone 32, applies stress on the terminal portions 26 of the radial arms of the sets 18 and 20 of haptics because, at rest, these terminal portions lie on a circle of diameter greater than the diameter of the equatorial zone of the capsular bag. This stress causes the arm to flex in the very localized connection end zone 22b of the arm, and it flexes about the flexing axis Z-Z′ because of the very small thickness in this zone. Because of the position of the contact edges 26 “in front” of the flexing lines 3-3′, this flexing causes the optic portion to be projected towards the posterior capsule 34, thereby pressing the posterior interface surface 14 again the posterior capsule 34. Because of the angle between the main portion 22a and its contact end 22c, while the connection portion is flexing, the contact end 22c of the arm comes to bear against the anterior peripheral portion 36 of the capsular bag. The main portion 22a occupies a position that makes a considerable angle relative to the optical plane P-P′, which angle is greater than 30°. Each arm thus has a contact end 22c bearing against the anterior portion of the capsular bag, and a main portion 22a presenting a large angle with the optical plane, thus causing the optic portion 10 to be pressed in stable and strong manner against the posterior capsule 34, thereby ensuring great axial stability for the implant.

In addition, as explained above, because the posterior interface surface 14 is pressed against the posterior capsule, this avoids cells proliferating on the capsular bag, where such proliferation can make the bag opaque. To reinforce this effect, the posterior interface surface 14 preferably presents a “square edge” 40 relative to the periphery 16 of the optic portion, thereby further increasing the effect of preventing cells from proliferating. Because of the small thickness of the connection end of each arm, thereby forming the above-described “step” 25, the square edge of the optic portion is continuous, since it is not interrupted in the connection zones between the periphery of the optic portion and the haptic arms.

FIG. 4 plots the curve of the axial displacement A of the implant optic as a function of compression C that depends on the difference between the diameter of the capsular bag and the outside diameter of the implant at rest. This curve shows that even for a non-negligible difference E compared with the theoretical diameter of the capsular bag, the axial displacement difference D is very small.

A second embodiment of the implant of the invention is described below with reference to FIGS. 2A and 2B. This embodiment includes an optic portion 10 that is identical to the optic portion of the implant 1A, and it includes a haptic portion. The haptic portion is constituted by four radial arms 40, 42, 44, and 46 that are angularly offset at 90° intervals. Each arm 40 to 46 has exactly the same configuration as the arm 22 described in detail with reference to FIG. 1A. In particular, the arm 40 has a connection end 40b of small section, a main portion 40a, a contact end 40c, and a contact edge 41.

The haptic portion of the implant also has four link pieces given respective references 48, 50, 52, and 54. These link pieces lie on a circle C2 as described above with reference to FIG. 1A. Each link piece 48 has two link ends 48a, 48b for the connection piece 48 which are connected to the arms 40 and 42 level with their bends 56. It should be observed that the link pieces 48 and 54 are of small right section, e.g. 0.25 mm×0.25 mm. As explained below with reference to FIG. 5, during flexing of the arms 40 to 46, after the implant has been put into place in the capsular bag, the connection pieces 48 must be capable of folding because of the reduction in the circular angular distance between the bends 56 in the two arms that are associated with any one link piece, e.g. the piece 48.

FIG. 5 shows that the radial arms 40 to 46 behave individually exactly like the arms of the implant shown in FIGS. 1A and 1B, with the contact ends 40c, 42c, etc. coming to bear against the residual anterior capsule. Under the effect of the ends of the arms moving towards each other, the link pieces 48 to 50 become deformed elastically and their middle portions 58 also come into contact with the equatorial portion of the capsular bag. This increases the contact area between the capsular bag and the haptic portion, and thus reduces the pressure applied to the capsular bag.

Naturally, it would not go beyond the invention if the haptic portion were to comprise only two arms that are diametrically opposite or three arms offset at 120° intervals.

It would also naturally not go beyond the invention for the contact ends of the haptic arms to be in line with the main portions thereof. Under such circumstances, the angle a between the main portion of an arm and the optical plane P-P′ could be very small, e.g. equal to 1° or 2°.

This is shown in FIGS. 6A and 6B. These figures show the optic portion 10′ with its peripheral edge 16. The haptic portion is constituted by two identical and diametrically opposite arms 60 and 62. Each arm comprises a main portion 60a, 62a and a connection end 60b, 62b having the same shape and the same characteristics as the connection ends of the other two embodiments. In other words, these connection ends define flexing lines Z-Z′.

In this embodiment, the haptic arms do not have “bent” contact ends. Consequently, the contact edge 64 of each arm is constituted by the end of the main portion 60a, 62a. Naturally, the contact edges 64 must satisfy the same conditions as specified for the first two embodiments.

1. A capsular bag intraocular implant comprising:
an optic portion of substantially circular shape defining an edge, an anterior interface surface, and a posterior interface surface; and
a haptic portion comprising at least two arms extending radially relative to the optic portion, comprising:
a main portion;
a connection end connected to the optic portion, said connection end having a thickness in the direction of the optical axis that is smaller than the thickness of the main portion so as to form a flexing line that is substantially tangential to the optic portion; and
a contact end presenting a contact edge for contacting the inside wall of the capsular bag, said contact edge being disposed on a circle (C1) that is concentric about the optic portion and of diameter greater than the diameter of the capsular bag, being not less than 10.5 mm;
said main portion of each arm forming an angle in a forward direction relative to the optical plane (P-P′) in such a manner that said flexing line is closer to the optical plane than to said contact edge;
whereby, when the implant is put into place in the capsular bag, the optic portion is displaced towards the posterior wall of the capsular bag by the arms turning about the flexing lines defined by their connection ends, under the effect of the stress applied by the capsular bag to the contact ends of the arms.

2. An intraocular implant according to claim 1, wherein said contact end of each arm is bent rearwards relative to the main portion of the arm, forming a bend in such a manner that the contact edge is closer to the optical plane of the optic portion than is the bend,
whereby, under the effect of the stress applied by the capsular bag, the connection ends come to bear against the anterior wall of the capsular bag.

3. An intraocular implant according to claim 2, wherein the angle b between the main portion of an arm and its contact end lies in the range 90° to 150°.

4. An intraocular implant according to claims 1, further comprising at least two connection pieces in the form of circular arcs concentric with the optic portion, each end of each connection piece being connected to an arm, said connection pieces being disposed on the same circle.

5. An intraocular implant according to claims 3, wherein the ends of the connection pieces are connected to the arms at the bends so that the contact ends of the arms extend beyond the circle on which said connection pieces are disposed.

6. An intraocular implant according to claim 4, wherein said haptic portion comprises four arms offset at 90° intervals, and four connection pieces.

7. An intraocular implant according to claim 4 wherein said haptic portion comprises four arms forming two pairs of arms, the arms in any one pair being offset from each other by an angle of less than 90°, the haptic portion also having two connection pieces, each connection piece interconnecting the two arms of a corresponding pair.

8. An intraocular implant according to claim 1, wherein the length of the connection end is shorter than the width of the main portion of the arm.

9. An intraocular implant according to claims 1, wherein the contact edge of each arm is in the form of an arc of a circle of diameter lying in the range 2.5 mm to 10.5 mm, whereby said edge is in contact with the capsular bag over a large zone.

10. An intraocular implant according to any one of claims 1, wherein the periphery of the posterior interface surface forms a “square” edge with the edge of the optic portion.

11. An intraocular implant according to claim 10, wherein the small thickness of the connection end creates a posterior “step” that extends said posterior “square” edge.

12. An intraocular implant according to claim 9, wherein the contact edge of each arm is in the form of an arc of a circle of diameter of 10 mm.
